(19) United States
(12) Patent Application Publication
(10) Pub. No.: US 2006/0088654 A1
(43) Pub. Date: Apr. 27, 2006

(54) Drug release coated stent

(75) Inventors: Ni Ding, San Jose, CA (US); Michael Helmus, Worcester, MA (US)

Correspondence Address: JONES DAY, 222 EAST 41ST ST, NEW YORK, NY, 10017, US

(73) Assignee: Boston Scientific Scimed, Inc.

(21) Appl. No.: 11/296,765

(22) Filed: Dec. 6, 2005

Related U.S. Application Data
(63) Continuation of application No. 10/022,607, filed on Dec. 17, 2001.
Application No. 10/022,607 is a continuation-in-part of application No. 09/079,645, filed on May 15, 1998, now abandoned.
Application No. 09/079,645 is a continuation of application No. 08/730,542, filed on Oct. 11, 1996, now abandoned.
Application No. 08/730,542 is a continuation of application No. 08/424,884, filed on Apr. 19, 1995, now abandoned.
(63) Continuation of application No. 10/022,607, filed on Dec. 17, 2001.
Application No. 10/022,607 is a continuation-in-part of application No. 09/012,443, filed on Jan. 23, 1998, now Pat. No. 6,358,556.
Application No. 09/012,443 is a division of application No. 08/663,490, filed on Jun. 13, 1996, now Pat. No. 5,837,313.
Application No. 08/663,490 is a continuation-in-part of application No. 08/526,273, filed on Sep. 11, 1995, now abandoned.
Application No. 08/526,273 is a continuation-in-part of application No. 08/424,884, filed on Apr. 19, 1995, now abandoned.

(51) Int. Cl. A61L 33/00

(52) U.S. Cl. 427/2.21; 427/2.25

(57) ABSTRACT

The present invention is directed to an expandable stent for implantation in a patient comprising a tubular metal body having open ends and a sidewall structure having openings therein and a coating disposed on a surface of said sidewall structure, said coating comprising a hydrophobic biostable elastomeric material and a biologically active material, wherein said coating continuously conforms to said structure in a manner that preserves said openings.

### CROSS-REFERENCE TO RELATED APPLICATION

The present application is a Continuation application of co-pending U.S. patent application Ser. No. 10/022,607, filed on Dec. 17, 2001, which is Continuation-In-Part of U.S. patent application Ser. No. 09/079,645, filed May 15, 1998, which is a Continuation of U.S. patent application Ser. No. 08/730,542, filed Oct. 11, 1996, abandoned, which is a FWC of U.S. patent application Ser. No. 08/424,884, filed Apr. 19, 1995, abandoned; and co-pending U.S. patent application Ser. No. 10/022,607, filed on Dec. 17, 2001, is also a Continuation-In-Part of U.S. patent application Ser. No. 09/012,443, filed Jan. 23, 1998, which is a Division of U.S. patent application Ser. No. 08/663,490, filed Jun. 13, 1996, U.S. Pat. No. 5,837,313, which is a Continuation-In-Part of U.S. patent application Ser. No. 08/526,273, filed Sep. 11, 1995, abandoned, which is a Continuation-In-Part of U.S. patent application Ser. No. 08/424,884, filed Apr. 19, 1995, abandoned, all portions of the above applications not contained in this application being deemed incorporated by reference for any purpose.

### BACKGROUND OF THE INVENTION

I. Field of the Invention

The present invention relates generally to therapeutic expandable stent prostheses for implantation in body lumens, e.g., vascular implantation and, more particularly, to a process for providing biostable elastomeric coatings on such stents which incorporate biologically active species having controlled release characteristics directly in the coating structure.

II. Related Art

In surgical or other related invasive medicinal procedures, the insertion and expansion of stent devices in blood vessels, urinary tracts or other difficult to access places for the purpose of preventing restenosis, providing vessel or lumen wall support or reinforcement and for other therapeutic or restorative functions has become a common form of long-term treatment. Typically, such prostheses are applied to a location of interest utilizing a vascular catheter, or similar transluminal device, to carry the stent to the location of interest where it is thereafter released to expand or be expanded in situ. These devices are generally designed as permanent implants which may become incorporated in the vascular or other tissue which they contact at implantation.

Stent devices of the self-expanding tubular type for transluminal implantation, then, are generally known. One type of such device includes a flexible tubular body which is composed of several individual flexible thread elements each of which extends in a helix configuration with the centerline of the body serving as a common axis. The elements have the same direction of winding but are displaced axially relative to each other and meet, under crossing a like number of elements also so axially displaced, but having the opposite direction of winding. This configuration provides a resilient braided tubular structure which assumes stable dimensions upon relaxation. Axial tension produces elongation and corresponding diameter contraction that allows the stent to be mounted on a catheter device and conveyed through the vascular system as a narrow elongated device. Once tension is relaxed in situ, the device at least substantially reverts to its original shape. Prostheses of the class including a braided flexible tubular body are illustrated and described in U.S. Pat. Nos. 4,655,771 and 4,954,126 to Wallsten and U.S. Pat. No. 5,061,275 to Wallsten et al.

The general idea of utilizing implanted stents to carry medicinal agents, such as thrombolytic agents, also has been proposed. U.S. Pat. No. 5,163,952 to Froix discloses a thermal memoried expanding plastic stent device which can be formulated to carry a medicinal agent by utilizing the material of the stent itself as an inert polymeric drug carrier. Pinchuk, in U.S. Pat. No. 5,092,877, discloses a stent of a polymeric material which may be employed with a coating associated with the delivery of drugs. Other patents which are directed to devices of the class utilizing bio-degradable or bio-sorbable polymers include Tang et al, U.S. Pat. No. 4,916,193, and MacGregor, U.S. Pat. No. 4,994,071. A patent to Sahatjian, U.S. Pat. No. 5,304,121, discloses a coating applied to a stent consisting of a hydrogel polymer and a preselected drug in which possible drugs include cell growth inhibitors and heparin. A further method of making a coated intravascular stent carrying a therapeutic material in which a polymer coating is dissolved in a solvent and the therapeutic material dispersed in the solvent and the solvent thereafter evaporated is described in European patent application 0623354 A1, published Nov. 9, 1994.

An article by Michael N. Helmus (a co-inventor of the present invention) entitled “Medical Device Design—A Systems Approach: Central Venous Catheters”, 22nd International Society for the Advancement of Material and Process Engineering Technical Conference (1990) relates to polymer/drug/membrane systems for releasing heparin. Those polymer/drug/membrane systems require two distinct layers of function.

The above cross-referenced application supplies an approach that provides long-term drug release, i.e., over a period of days or even months, incorporated in a controlled-release system. The present invention provides an expandable coated stent having a sidewall having openings therein and a coating on a surface of the sidewall structure, wherein the coating continuously conforms to the structure in a manner that preserves the openings, particularly when the stent is expanded.

Polymeric stents, although effective, generally cannot equal the mechanical properties of metal stents of like thickness and weave. For example, in keeping a vessel open, a metallic stent is generally superior because stents braided of even relatively fine metal can provide a large amount of strength to resist inwardly directed circumferential pressure. In order for a polymer material to provide comparable strength characteristics, a much thicker-walled structure or heavier, denser filament weave is required. This, in turn, reduces the cross-sectional area available for flow through the stent and/or reduces the relative amount of open space available in the structure. In addition, when applicable, it is usually more difficult to load such a stent onto catheter delivery systems for conveyance through the vascular system of the patient to the site of interest.

It will be noted, however, that while certain types of stents such as braided metal stents may be superior to others for some applications, the present invention is not limited in that respect and may be used to coat a wide variety of devices. The present invention also applies, for example, to the class of stents that are not self-expanding including those which can be expanded, for instance, with a balloon. Polymeric stents, of all kinds can be coated using the process. Thus, regardless of detailed embodiments the use of the invention is not considered to be limited with respect either to stent design or materials of construction.

Accordingly, it is a primary object of the present invention to provide an expandable coated stent having a sidewall having openings therein and a coating on a surface of the sidewall structure, wherein the coating continuously conforms to the structure in a manner that preserves the openings, particularly when the stent expanded.

Still another object of the present invention is to provide an expandable coated stent having a sidewall having openings therein and a coating on a surface of the sidewall structure, wherein the openings are substantially free of webbing.

Other objects and advantages of the present invention will become apparent to those skilled in the art upon familiarization with the specification and appended claims.

### SUMMARY OF THE INVENTION

The present invention provides a relatively thin layer of biostable elastomeric material in which an amount of biologically active material is dispersed therein as a coating on the surfaces of a deployable expandable stent prosthesis. The preferred stent to be coated is a self-expanding, open-ended tubular stent prosthesis. Although other materials, including polymer materials, can be used, in the preferred embodiment, the tubular body is formed of an open braid of fine single or polyfilament metal wire which flexes without collapsing and readily axially deforms to an elongate shape for transluminal insertion via a vascular catheter. The stent resiliently attempts to resume predetermined stable dimensions upon relaxation in situ.

The coating is preferably applied as a mixture, solution or suspension of polymeric precursor and finely divided biologically active species dispersed in an organic vehicle or a solution or partial solution of such species in a solvent or vehicle for the polymer and/or biologically active species. For the purpose of this application, the term “finely divided” means any type or size of included material from dissolved molecules through suspensions, colloids and particulate mixtures. The active material is dispersed in a carrier material which may be the polymer, a solvent, or both. The coating is preferably applied as a plurality of relatively thin layers sequentially applied in relatively rapid sequence and is preferably applied with the stent in a radially expanded state. In some applications the coating may further be characterized as a composite initial or tie coat and a composite top coat. The coating thickness ratio of the top coat to the tie coat may vary with the desired effect and/or the elution system. Typically these are of different formulations.

The coating may be applied by dipping or spraying using evaporative solvent materials of relatively high vapor pressure to produce the desired viscosity and quickly establish coating layer thicknesses. The preferred process is predicated on reciprocally spray coating a rotating radially expanded stent employing an air brush device. The coating process enables the material to adherently conform to and cover the entire surface of the filaments of the open structure of the stent but in a manner such that the open lattice nature of the structure of the braid or other pattern is preserved, in the coated device.

The coating is exposed to room temperature ventilation for a predetermined time (possibly one hour or more) for solvent vehicle evaporation. Thereafter the polymer material is cured at room temperature or elevated temperatures. Curing is defined as the process of converting the elastomeric or polymeric material into the finished or useful state by the application of heat and/or chemical agents which induce physico-chemical changes.

The ventilation time and temperature for cure are determined by the particular polymer involved and particular drugs used. For example, silicone or polysiloxane materials (such as polydimethylsiloxane) have been used successfully. These materials are applied as polymer precursors in the coating composition and must thereafter be cured. The preferred species have a relatively low cure temperatures and are known as a room temperature vulcanizable (RTV) materials. Some polydimethylsiloxane materials can be cured, for example, by exposure to air at about 90° C. for a period of time such as 16 hours. A curing step may be implemented both after application of the tie or a certain number of lower layers and the top layers or a single curing step used after coating is completed.

The coated stents may thereafter be subjected to a postcure sterilization process which includes an inert gas plasma treatment, and then exposure to gamma radiation, electron beam, ethylene oxide (ETO) or steam sterilization may also be employed.

In the plasma treatment, unconstrained coated stents are placed in a reactor chamber and the system is purged with nitrogen and a vacuum applied to 20 mTorr. Thereafter, inert gas (argon, helium or mixture of them) is admitted to the reaction chamber for the plasma treatment. A highly preferred method of operation consists of using argon gas, operating at a power range from 200 to 400 watts, a flow rate of 150-650 standard ml per minute, which is equivalent to about 100-450 mTorr, and an exposure time from 30 seconds to about 5 minutes. The stents can be removed immediately after the plasma treatment or remain in the argon atmosphere for an additional period of time, typically five minutes.

After the argon plasma pretreatment, the coated and cured stents are subjected to gamma radiation sterilization nominally at 2.5-3.5 Mrad. The stents enjoy full resiliency after radiation whether exposed in a constrained or non-constrained status. It has been found that constrained stents subjected to gamma sterilization without utilizing the argon plasma pretreatment lose resiliency and do not recover at a sufficient or appropriate rate.

The elastomeric material that forms a major constituent of the stent coating should possess certain properties. It is preferably a suitable hydrophobic biostable elastomeric material which does not degrade and which minimizes tissue rejection and tissue inflammation and one which will undergo encapsulation by tissue adjacent the stent implantation site. Polymers suitable for such coatings include silicones (e.g., polysiloxanes and substituted polysiloxanes), polyurethanes, thermoplastic elastomers in general, ethylene vinyl acetate copolymers, polyolefin, elastomers, and EPDM rubbers. The above-referenced materials are considered hydrophobic with respect to the contemplated environment of the invention.

Agents suitable for incorporation include antithrobotics, anticoagulants, antiplatelet agents, thorombolytics, antiproliferatives, antinflammatories, agents that inhibit hyperplasia and in particular restenosis, smooth muscle cell inhibitors, growth factors, growth factor inhibitors, cell adhesion inhibitors, cell adhesion promoters and drugs that may enhance the formation of healthy neointimal tissue, including endothelial cell regeneration. The positive action may come from inhibiting particular cells (e.g., smooth muscle cells) or tissue formation (e.g., fibromuscular tissue) while encouraging different cell migration (e.g., endothelium) and tissue formation (neointimal tissue).

The preferred materials for fabricating the braided stent include stainless steel, tantalum, titanium alloys including nitinol (a nickel titanium, thermomemoried alloy material), and certain cobalt alloys including cobalt-chromium-nickel alloys such as Elgiloy® and Phynox®. Further details concerning the fabrication and details of other aspects of the stents themselves, may be gleaned from the above referenced U.S. Pat. Nos. 4,655,771 and 4,954,126 to Wallsten and U.S. Pat. No. 5,061,275 to Wallsten et al. To the extent additional information contained in the above-referenced patents is necessary for an understanding of the present invention, they are deemed incorporated by reference herein.

Various combinations of polymer coating materials can be coordinated with biologically active species of interest to produce desired effects when coated on stents to be implanted in accordance with the invention. Loadings of therapeutic materials may vary. The mechanism of incorporation of the biologically active species into the surface coating, and egress mechanism depend both on the nature of the surface coating polymer and the material to be incorporated. The mechanism of release also depends on the mode of incorporation. The material may elute via interparticle paths or be administered via transport or diffusion through the encapsulating material itself.

For the purposes of this specification, “elution” is defined as any process of release that involves extraction or release by direct contact of the material with bodily fluids through the interparticle paths connected with the exterior of the coating.

“Transport” or “diffusion” are defined to include a mechanism of release in which the material released traverses through another material.

The desired release rate profile can be tailored by varying the coating thickness, the radial distribution (layer to layer) of bioactive materials, the mixing method, the amount of bioactive material, the combination of different matrix polymer materials at different layers, and the crosslink density of the polymeric material. The crosslink density is related to the amount of crosslinking which takes place and also the relative tightness of the matrix created by the particular crosslinking agent used. This, during the curing process, determines the amount of crosslinking and so the crosslink density of the polymer material. For bioactive materials released from the crosslinked matrix, such as heparin, a denser crosslink structure will result in a longer release time and reduced burst effect.

It will also be appreciated that an unmedicated silicone top layer provides an advantage over drug containing top coat. Its surface is non-porous and smooth, which may be less thrombogeneous and may reduce the chance to develop calcification, which occurs most often on the porous surface.

In one embodiment of the present application, an expandable stent, such as a self-expandable stent, for implantation in a patient includes a tubular metal body having open ends and a sidewall structure having openings therein and a coating disposed on a surface of the sidewall structure. The coating comprises a hydrophobic biostable elastomeric material and a biologically active material. The coating continuously conforms to the structure in a manner that preserves the openings, such as when the stent is expanding. The coating may be about 20 to about 200 μm in thickness or about 75 to about 200 μm in thickness.

The coating can be applied to the surface of the sidewall structure by spraying a coating composition comprising a mixture of finely divided biologically active species and an about 4to 6 w/v % dispersion of uncured hydrophobic biostable elastomeric material in a solvent. The coating may be applied with the stent fully expanded. Also, the coating may be applied with the stent rotated.

The metal can be selected from the group consisting of stainless steel, titanium alloys, tantalum, and cobalt-chrome alloys. The biostable elastomeric material may be selected from the group consisting of polysiloxanes, polyurethanes, thermoplastic elastomers, ethylene vinyl acetate copolymers, polyolefin elastomers, ethylene-propylene terpolymer rubbers and combinations thereof. In a certain embodiment, the biostable elastomeric material is a polysiloxane and the biologically active species is selected from the group consisting of heparin and dexamethasone.

In another embodiment, an expandable stent, such as a self-expandable stent, for implantation in a patient includes a tubular metal body having open ends and a sidewall structure having openings therein and a coating disposed on a surface of the sidewall structure. The coating comprises a hydrophobic biostable elastomeric material and a biologically active material. The openings are substantially free of webbing. The coating may be about 20 to about 200 μm in thickness or about 75 to about 200 μm in thickness.

The coating can be applied to the surface of the sidewall structure by spraying a coating composition comprising a mixture of finely divided biologically active species and an about 4 to 6 w/v % dispersion of uncured hydrophobic biostable elastomeric material in a solvent. The coating may be applied with the stent fully expanded. Also, the coating may be applied with the stent rotated.

The metal can be selected from the group consisting of stainless steel, titanium alloys, tantalum, and cobalt-chrome alloys. The biostable elastomeric material may be selected from the group consisting of polysiloxanes, polyurethanes, thermoplastic elastomers, ethylene vinyl acetate copolymers, polyolefin elastomers, ethylene-propylene terpolymer rubbers and combinations thereof. In a certain embodiment, the biostable elastomeric material is a polysiloxane and the biologically active species is selected from the group consisting of heparin and dexamethasone.

Also, the openings may be substantially in the shape of a parallelogram with first and third sides that are substantially parallel and second and fourth sides that are substantially parallel, wherein the openings are substantially free of webbing such that any imaginary line extended orthogonally from the first side to the third side does not intersect the coating extending between the second and fourth sides.

In yet another embodiment, a self-expandable stent for implantation in a patient comprises a tubular metal body having open ends and a sidewall structure having openings therein and a coating of about 75 to about 200 μm in thickness on a surface of the sidewall structure. The coating comprises a biologically active material and a hydrophobic biostable elastomeric material selected from the group consisting of polysiloxanes, polyurethanes, thermoplastic elastomers, ethylene vinyl acetate copolymers, polyolefin elastomers, ethylene-propylene terpolymer rubbers and combinations thereof. The coating continuously conforms to the structure in a manner that preserves the openings, such as when the stent is expanded. The coating may also continuously conform to the structure in a manner that the openings are substantially free of webbing.

The coating may be applied to the surface of the sidewall structure while the stent is fully expanded and rotated by spraying, with an air brush with its pressure adjusted to from about 15 to about 25 psi, a coating composition comprising a mixture of finely divided biologically active species and a dispersion of uncured hydrophobic biostable elastomeric material in a solvent and then cured.

The stent may be rotated at speeds in the range of about 30 to about 50 rpm. Also, the coating composition may be sprayed at a spray nozzle flow rate in the range of about 4 to about 10 ml. In addition, the coating can comprise more than one coating layer.

### BRIEF DESCRIPTION OF THE DRAWINGS

In the drawings, wherein like numerals designate like parts throughout the same:

FIGS. 1 and 1A depict greatly enlarged views of a fragment of a medical stent for use with the coating of the invention;

FIGS. 2A and 2B depict a view of a stent section as pictured in FIGS. 1 and 1A as stretched or elongated for insertion;

FIG. 3 is a light microscopic photograph of a typical uncoated stent structure configuration (20×);

FIG. 4A is a scanning electron microscope photograph (SEM) of a heparin containing poly siloxane coating on a stent in accordance with the invention (×20) after release of heparin into buffer for 49 days;

FIG. 4B is a higher powered scanning electron microscopic photograph (SEM) of the coating of FIG. 4A (×600);

FIG. 5A is another scanning electron microscopic photograph (SEM) of a different stent coated with coating as produced with heparin incorporated into the polysiloxane (×20);

FIG. 5B is an enlarged scanning electron microscopic photograph (SEM) of the coating of FIG. 5B (×600);

FIG. 6A is a light microscopic picture (×17.5) of a histologic cross-section of a silicone/heparin coated stent implanted in a swine coronary for 1 day;

FIG. 6B depicts a pair of coated filaments of the stent of FIG. 6A (×140) showing heparin provided in silicone;

FIG. 7A is a scanning electron microscope photograph (SEM) that depicts a polysiloxane coating containing 5% dexamethasone (×600);

FIG. 7B depicts the coating of FIG. 7A (SEM ×600) after dexamethasone release in polyethylene glycol (PEG 400/H₂O) for three months;

FIG. 8 is a plot showing the total percent heparin released over 90 days from a coated stent in which the coated layer is 50% heparin (based on the total weight of the coating) in a silicone polymer matrix; release took place in phosphoric buffer (pH=7.4) at 37° C.; and

FIG. 9 is a plot of the total percent dexamethasone released over 100 days for two percentages of dexamethasone in silicon coated stents; release took place in polyethylene glycol (PEG), MW=400 (PEG 400/H₂O, 40/60, vol/vol) at 37° C.

FIG. 10 is a schematic flow diagram illustrating the steps of the process of the invention;

FIG. 11 represents a release profile for a multi-layer system showing the percentage of heparin released over a two-week period;

FIG. 12 represents a release profile for a multi-layer system showing the relative release rate of heparin over a two-week period;

FIG. 13 illustrates a profile of release kinetics for different drug loadings at similar coating thicknesses illustrating the release of heparin over a two-week period;

FIG. 14 illustrates drug elution kinetics at a given loading of heparin over a two-week period at different coating thicknesses; and

FIG. 15 illustrates the release kinetics in a coating having a given tie-layer thickness for different top coat thicknesses in which the percentage heparin in the tie coat and top coats are kept constant (37.5% heparin in tie-coat with the same tie-coat thickness and 16.7% heparin in top-coat).

### DETAILED DESCRIPTION

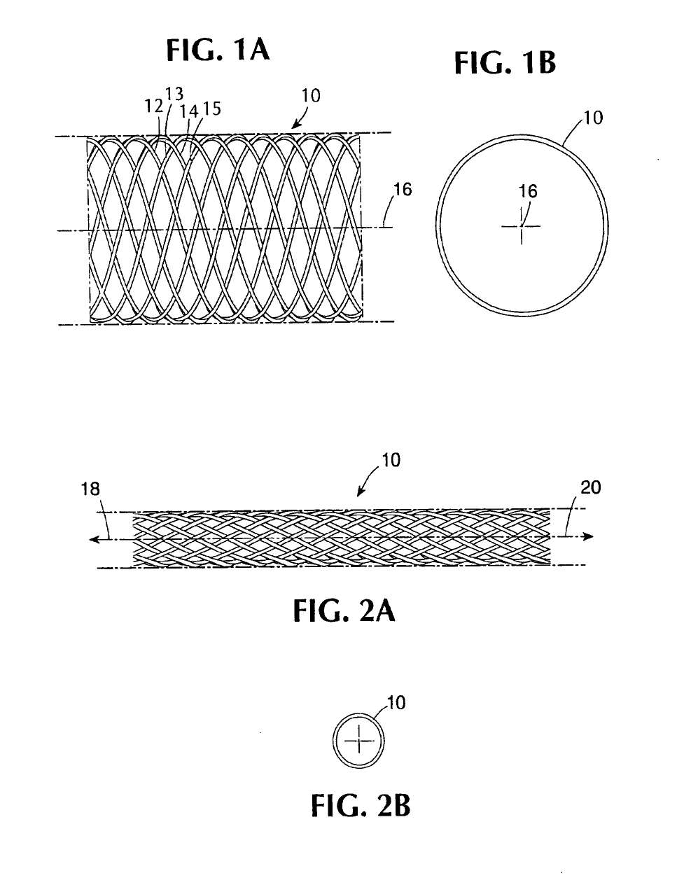

A type of stent device of one class designed to be utilized in combination with coatings in the present invention is shown diagrammatically in a side view and an end view, respectively contained in FIGS. 1A and 1B. FIG. 1A shows a section of a generally cylindrical tubular body 10 having a mantle surface formed by a number of individual thread elements 12, 14 and 13, 15, etc. of these elements, elements 12, 14, etc. extend generally in an helix configuration axially displaced in relation to each other but having center line 16 of the body 10 as a common axis. The other elements 13, 15, likewise axially displaced, extend in helix configuration in the opposite direction, the elements extending in the two directions crossing each other in the manner indicated in FIG. 1A. A tubular member so concerned and so constructed can be designed to be any convenient diameter, it being remembered that the larger the desired diameter, the larger the number of filaments of a given wire diameter (gauge) having common composition and prior treatment required to produce a given radial compliance.

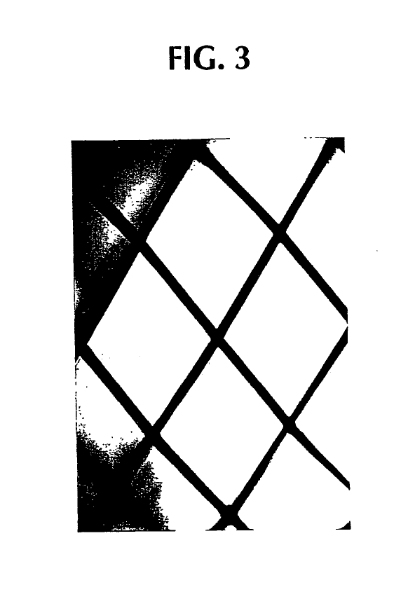

The braided structure further characteristically readily elongates upon application of tension to the ends axially displacing them relative to each other along center line 16 and correspondingly reducing the diameter of the device. This is illustrated in FIGS. 2A and 2B in which a segment of the device 10 of FIGS. 1A and 1B has been elongated by moving the ends 18 and 20 away from each other in the direction of the arrows. Upon the release of the tension on the ends, the structure 10, if otherwise unrestricted, will reassume the relaxed or unloaded configuration of FIGS. 1A and 1B.

The elongation/resumption characteristic flexibility of the stent device enables it to be slipped or threaded over a carrying device while elongated for transportation through the vascular or other relevant internal luminal system of a patient to the site of interest where it can be axially compressed and thereby released from the carrying mechanism, often a vascular catheter device. At the site of interest, it assumes an expanded condition held in place by mechanical/frictional pressure between the stent and the lumen wall against which it expands.

The elongation, loading, transport and deployment of such stents is well known and need not be further detailed here. It is important, however, to note that when one contemplates coatings for such a stent in the manner of the present invention, an important consideration resides in the need to utilize a coating material having elastic properties compatible with the elastic deforming properties residing in the stent that it coats. The material of the stent should be rigid and elastic but not plastically deformable as used. As stated above, the preferred materials for fabricating the metallic braided stent include stainless steel, tantalum, titanium alloys including nitinol and certain cobalt-chromium alloys. The diameter of the filaments may vary but for vascular devices, up to about 10 mm in diameter is preferable with the range 0.01 to 0.05 mm.

Drug release surface coatings on stents in accordance with the present invention can release drugs over a period of time from days to months and can be used, for example, to inhibit thrombus formation, inhibit smooth muscle cell migration and proliferation, inhibit hyperplasia and restenosis, and encourage the formation of health neointimal tissue including endothelial cell regeneration. As such, they can be used for chronic patency after an angioplasty or stent placement. It is further anticipated that the need for a second angioplasty procedure may be obviated in a significant percentage of patients in which a repeat procedure would otherwise be necessary. A major obstacle to the success of the implant of such stents, of course, has been the occurrence of thrombosis in certain arterial applications such as in coronary stenting. Of course, antiproliferative applications would include not only cardiovascular but any tubular vessel that stents are placed including urologic, pulmonary and gastro-intestinal.

Various combinations of polymer coating materials can be coordinated with the braided stent and the biologically active agent of interest to produce a combination which is compatible at the implant site of interest and controls the release of the biologically active species over a desired time period. Preferred coating polymers include silicones (poly siloxanes), polyurethanes, thermoplastic elastomers in general, ethylene vinyl acetate copolymers, polyolefin rubbers, EPDM rubbers, and combinations thereof.

Specific embodiments of the present invention include those designed to elute heparin to prevent thrombosis over a period of weeks or months or to allow the diffusion or transport of dexamethasone to inhibit fibromuscular proliferation over a like period of time. Of course, other therapeutic substances and combinations of substances are also contemplated. The invention may be implanted in a mammalian system, such as in a human body.

The heparin elution system is preferably fabricated by taking finely ground heparin crystal, preferably ground to an average particle size of less than 10 microns, and blending it into a liquid, uncured poly siloxane/solvent material in which the blend (poly siloxane plus heparin) contains from less than 10% to as high as 80% heparin by weight with respect to the total weight of the material and typically the layer is between 10% and 45% heparin.

This material is diluted with a solvent and utilized to coat a metallic braided stent, which may be braided cobalt chromium alloy wire, in a manner which applies a thin, uniform coating (typically between 20 and 200 microns in thickness) of the heparin/polymer mixture on the surfaces of the stent. The polymer is then heat cured, or cured using low temperature thermal initiators (<100° C.) in a room temperature vulcanization (RTV) process in situ on the stent to evaporate the solvent, typically tetrahydrofuran (TEF). The heparin forms interparticle paths in the silicone sufficiently interconnected to allow slow but substantially complete subsequent elution. The ultrafine particle size utilized allows the average pore size to be very small such that elution may take place over weeks or even months.

A coating containing dexamethasone is produced in a somewhat different manner. A poly siloxane material is also the preferred polymeric material. Nominally an amount equal to 0.4% to about 45% of the total weight of the layer of dexamethasone is used.

The dexamethasone drug is dissolved in a solvent, e.g., THF first. The solution is then blended into liquid uncured poly siloxane/solvent (xylene, THF, etc.) vehicle precursor material. Since the dexamethasone is also soluble in the solvent for the polysiloxane, it dissolves into the mixture. The coating is then applied to the stent and upon application, curing and drying, including evaporation of the solvent, the dexamethasone remains dispersed in the coating layer. It is believed that the coating is somewhat in the nature of a solid solution of recrystallized particles of dexamethasone in silicone rubber. Dexamethasone, as a rather small molecule, however, does not need gross pores to elute and may be transported or diffused outward through the silicone material over time to deliver its anti-inflammatory medicinal effects.

The coatings can be applied by dip coating or spray coating or even, in some cases, by the melting of a powdered form in situ or any other technique to which the particular polymer/biologically active agent combination is well suited.

It will be understood that a particularly important aspect of the present invention resides in the technology directed to the incorporation of very fine microparticles or colloidal suspensions of the drug into the polymer matrix. In the case of a crystalline drug, such as heparin, the drug release is controlled by the network the drug forms in the polymer matrix, the average particulate size controlling the porosity and so the ultimate elution rate.

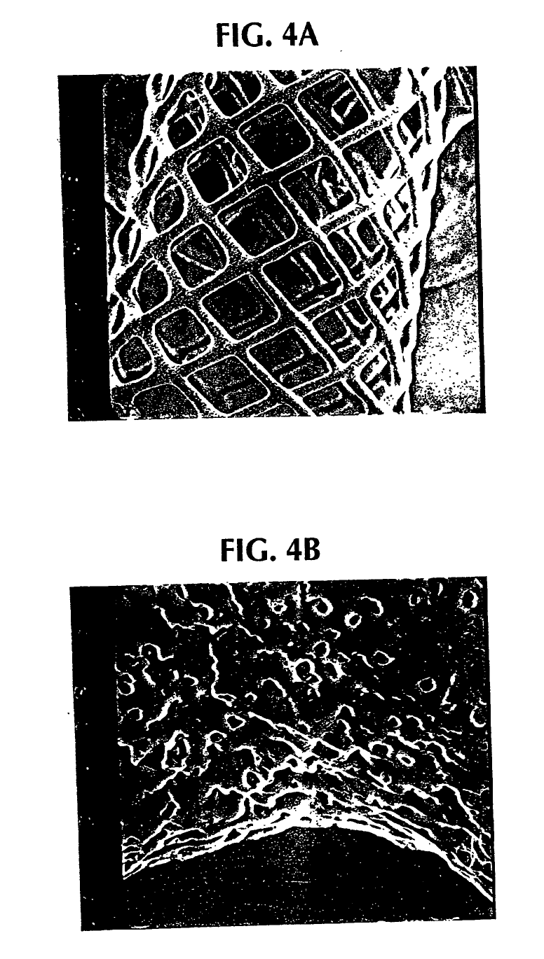

FIG. 4A depicts a stent which has been spray coated with a solvent containing a cured polysilicone material including an amount of heparin crystals to provide a thin, uniform coating on all surfaces of the stent. The coated stent was cured at 150° C. for 18 minutes; The sample was eluted in PBS for 49 days at 37° C. and the stent was rinsed in ethanol prior to taking the scanning electron microscope picture of FIG. 4A. FIG. 4B shows a greatly enlarged (600×) scanning electron microscope photograph (SEM) of a portion of the coating of FIG. 4A in which the microporosity is evident. The coating thickness may vary but is typically from about 75 to about 200 microns.

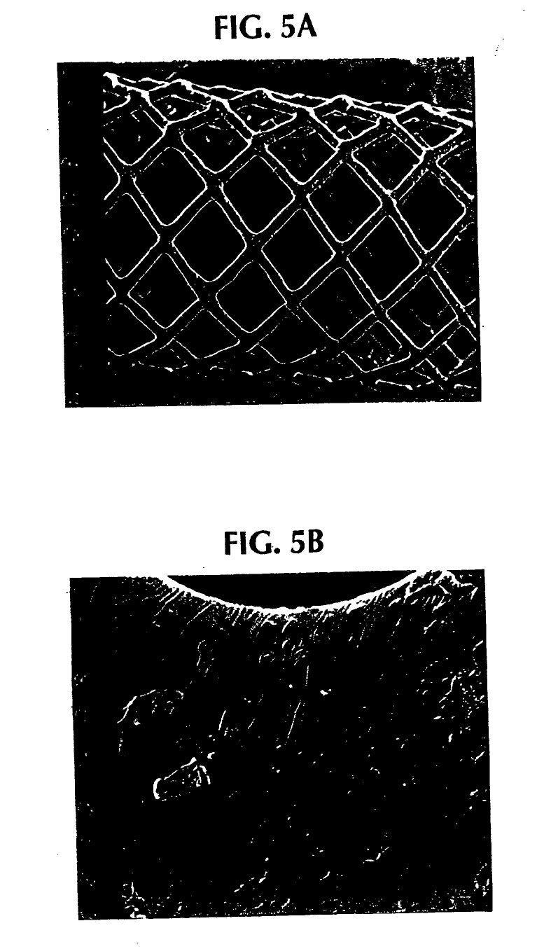

FIGS. 5A and 5B show scanning electron microscope photographs of a heparin containing polysiloxane stent. The Figure shows the coating prior to elution of the heparin. The coating was cured at 150 for 18 minutes. FIG. 5B is greatly enlarged photograph (SEX) of a fragment of the coated surface of FIG. 5A showing the substantially non-porous surface prior to elution.

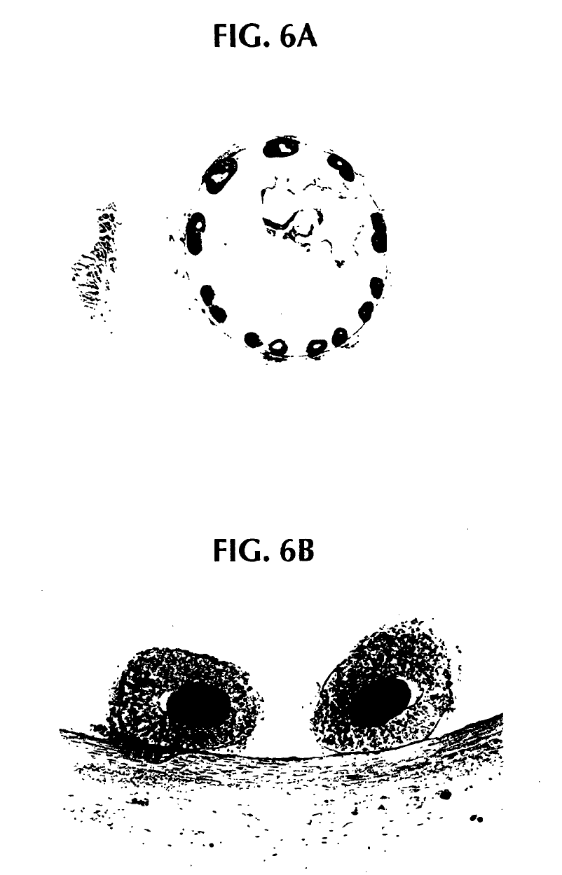

FIGS. 6A and 6B show the posture of a stent in accordance with the invention as implanted in a swine coronary. The blemish shown in FIG. 6A represents a histological artifact of unknown origin. As can be seen in FIG. 6B, a large number of heparin particles are contained in the silicone material.

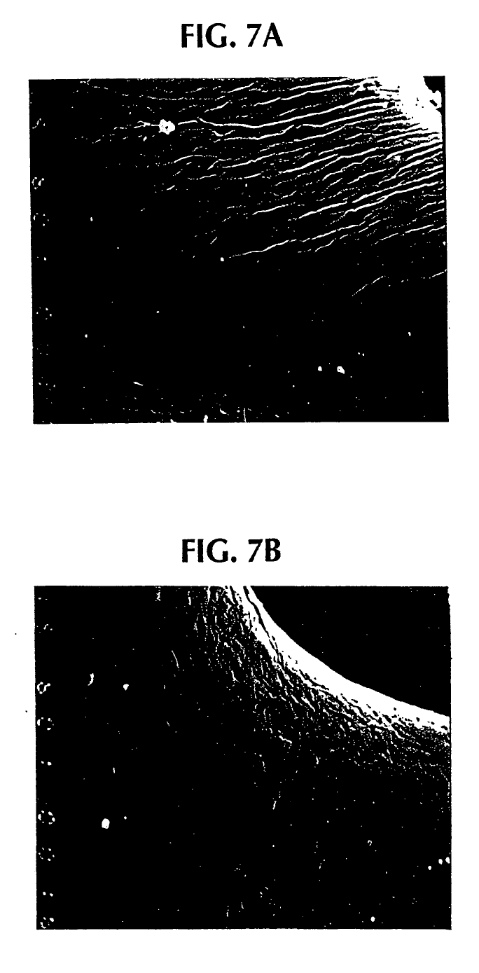

The substantially non-porous surface of FIG. 7A typically occurs with an incorporation of an amount of non-particulate material such as dexamethasone which partially or entirely dissolves in the solvent for the poly siloxane prior to coating and cure. Upon curing of the polymer and evaporation of the solvent, depending on the loading of dexamethasone, the dexamethasone reprecipitates in a hydrophobic crystalline form containing dendrite or even elongated hexagonal crystals approximately 5 microns in size.

As can be seen in FIG. 7B, even after release of the incorporated material or three months, the coating surface remains substantially non-porous indicating the transport or diffusion of the drug outward through the silicone material neither requires nor produces gross pores. The dexamethasone is incorporated in its more hydrophobic form rather than in one of the relatively more hydrophilic salt forms such as in a phosphate salt, for example.

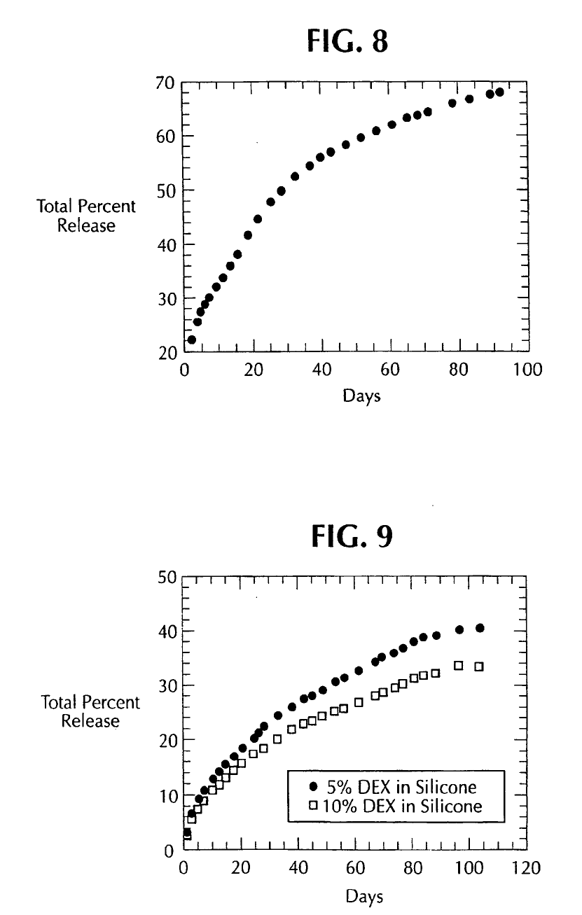

FIGS. 8 and 9 depict plots of total percent drug release related to long-term drug release stent coating layers. FIG. 8 depicts the release of heparin from a 50% heparin loading in silicone. The silicone was cured at 90° C. for 16 hours. The heparin release took place in a phosphoric buffer (pH=7.4) for 90 days at 37° C. The heparin concentration in the phosphoric buffer was analyzed by Azure A assay.

FIG. 9 depicts a graphical analysis, similar to that depicted for heparin in FIG. 8, for the release of dexamethasone at two different concentrations, i.e., 5% and 10% in silicone polymer. The coated stents were cured at 150° C. for 20 minutes and the release took place in a polyethylene glycol (PEG), MW=400/water solution at 37° C. ((PEG 400/H₂O) (40/60, vol/vol)). The dexamethasone concentrations were analyzed photometrically at 241 μm.

FIGS. 8 and 9 illustrate possible stent coating layers of polymer/bioactive species combinations for long-term release. As stated above, the release rate profile can be altered by varying the amount of active material, the coating thickness, the radial distribution of bioactive materials, the mixing method, and the crosslink density of the polymer matrix. Sufficient variation is possible such that almost any reasonable desired profile can be simulated.

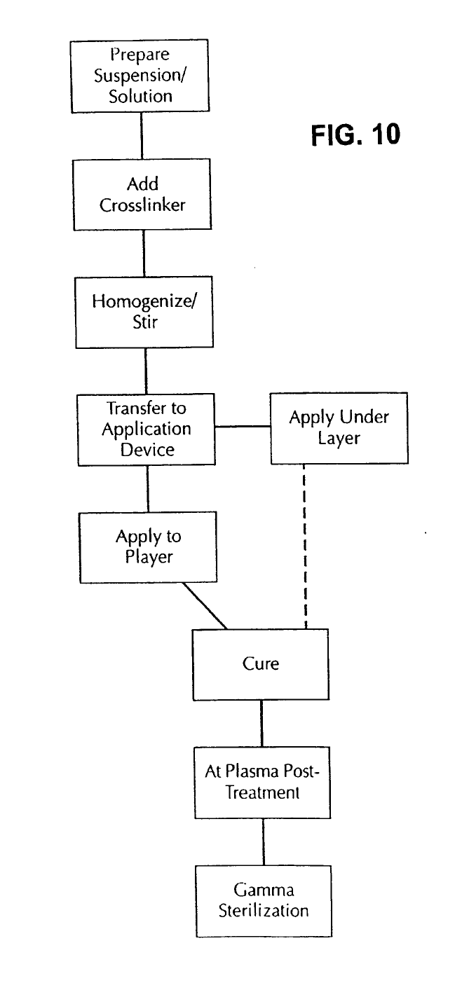

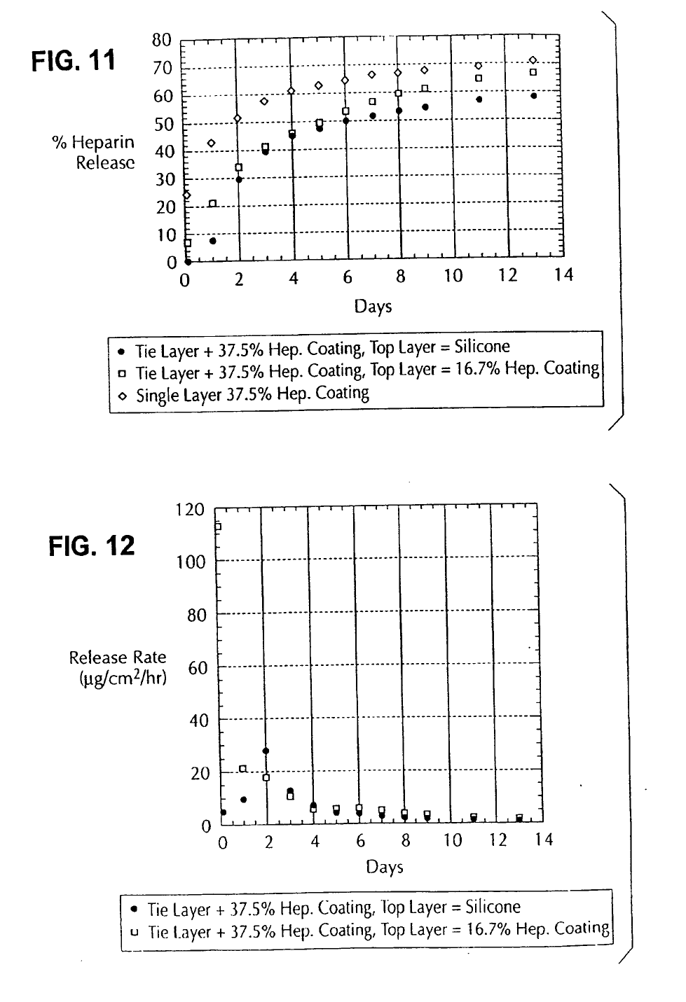

According to the present invention, the stent coatings incorporating biologically active materials for timed delivery in situ in a body lumen of interest are preferably sprayed in many thin layers from prepared coating solutions or suspensions. The steps of the process are illustrated generally in FIG. 10. The coating solutions or suspensions are prepared at 10 as will be described later. The desired amount of crosslinking agent is added to the suspension/solution as at 12 and material is then agitated or stirred to produce a homogenous coating composition at 14 which is thereafter transferred to an application container or device which may be a container for spray painting at 16. Typical exemplary preparations of coating solutions that were used for heparin and dexamethasone appear next.

General Preparation of Heparin Coating Composition

Silicone was obtained as a polymer precursor in solvent (xylene) mixture. For example, a 35% solid silicone weight content in xylene was procured from Applied Silicone, Part #40,000. First, the silicone-xylene mixture was weighed. The solid silicone content was determined according to the vendor's analysis. Precalculated amounts of finely divided heparin (2-6 microns) were added into the silicone, then tetrahydrofuron (THF) HPCL grade (Aldrich or EM) was added. For a 37.5% heparin coating, for example: W_{silicone}=5 g; solid percent=35%; Wₕₑₚ=5×0.35×0.375/(0.625)=1.05 g. The amount of THF needed (44 ml) in the coating solution was calculated by using the equation W_{silicone solid}/Va_{THF}=0.04 for a 37.5% heparin coating solution). Finally, the manufacturer crosslinker solution was added by using Pasteur P-pipet. The amount of crosslinker added was formed to effect the release rate profile. Typically, five drops of crosslinker solution were added for each five grams of silicone-xylene mixture. The crosslinker may be any suitable and compatible agent including platinum and peroxide based materials. The solution was stirred by using the stirring rod until the suspension was homogenous and milk-like. The coating solution was then transferred into a paint jar in condition for application by air brush.

General Preparation of Dexamethasone Coating Composition

Silicone (35% solution as above) was weighed into a beaker on a Metler balance. The weight of dexamethasone free alcohol or acetate form was calculated by silicone weight multiplied by 0.35 and the desired percentage of dexamethasone (1 to 40%) and the required amount was then weighed. Example: W_{silicone}=5 g; for a 10% dexamethasone coating, W_{dex}=5×0.35×0.1/0.9=0.194 g and THF needed in the coating solution calculated. W_{silicone solid}/V_{THF}=0.06 for a 10% dexamethasone coating solution. Example: W_{silicone}=5 g; V_{THF}=5×0.35/0.06=29 ml. The dexamethasone was weighed in a beaker on an analytical balance and half the total amount of THF was added. The solution was stirred well to ensure full dissolution of the dexamethasone. The stirred DEX-THF solution was then transferred to the silicone container. The beaker was washed with the remaining THF and this was transferred to the silicone container. The crosslinker was added by using a Pasteur pipet. Typically, five drops of crosslinker were used for five grams of silicone.

The application of the coating material to the stent was quite similar for all of the materials and the same for the heparin and dexamethasone suspensions prepared as in the above Examples. The suspension to be applied was transferred to an application device, typically a paint jar attached to an air brush, such as a Badger Model 150, supplied with a source of pressurized air through a regulator (Norgren, 0-160 psi). Once the brush hose was attached to the source of compressed air downstream of the regulator, the air was applied. The pressure was adjusted to approximately 15-25 psi and the nozzle condition checked by depressing the trigger.

While any appropriate method can be used to secure the stent for spraying, rotating fixtures were utilized successfully in the laboratory. Both ends of the relaxed stent were fastened to the fixture by two resilient retainers, commonly alligator clips, with the distance between the clips adjusted so that the stent remained in a relaxed, unstretched condition. The rotor was then energized and the spin speed adjusted to the desired coating speed, nominally about 40 rpm. With the stent rotating in a substantially horizontal plane, the spray nozzle was adjusted so that the distance from the nozzle to the stent was about 2-4 inches and the composition was sprayed substantially horizontally with the brush being directed along the stent from the distal end of the stent to the proximal end and then from the proximal end to the distal end in a sweeping motion at a speed such that one spray cycle occurred in about three stent rotations. Typically a pause of less than one minute, normally about one-half minute, elapsed between layers. Of course, the number of coating layers did and will vary with the particular application. For example, for a coating level of 3-4 mg of heparin per cm² of projected area, 20 cycles of coating application are required and about 30 ml of solution will be consumed for a 3.5 mm diameter by 14.5 cm long stent.

The rotation speed of the motor, of course, can be adjusted as can the viscosity of the composition and the flow rate of the spray nozzle as desired to modify the layered structure. Generally, with the above mixes, the best results have been obtained at rotational speeds in the range of 30-50 rpm and with a spray nozzle flow rate in the range of 4-10 ml of coating composition per minute, depending on the stent size. It is contemplated that a more sophisticated, computer-controlled coating apparatus will successfully automate the process demonstrated as feasible in the laboratory.

Several applied layers make up what is called the tie layer as at 18 and thereafter additional upper layers, which may be of a different composition with respect to bioactive material, the matrix polymeric materials and crosslinking agent, for example, are applied as the top layer as at 20. The application of the top layer follows the same coating procedure as the tie layer with the number and thickness of layers being optional. Of course, the thickness of each layer can be adjusted by adjusting the speed of rotation of the stent and the spraying conditions. Generally, the total coating thickness is controlled by the number of spraying cycles or thin coats which make up the total coat.

As shown at 22 in FIG. 10, the coated stent is thereafter subjected to a curing step in which the polymer precursor and crosslinking agents cooperate to produce a cured polymer matrix containing the biologically active species. The curing process involves evaporation of the solvent xylene, THF, etc. and the curing and crosslinking of the polymer. Certain silicone materials can be cured at relatively low temperatures, (i.e. RT-50° C.) in what is known as a room temperature vulcanization (RTV) process. More typically, however, the curing process involves higher temperature curing materials and the coated stents are put into an oven at approximately 90° C. or higher for approximately 16 hours. The temperature may be raised to as high as 150° C. for dexamethasone containing coated stents. Of course, the time and temperature may vary with particular silicones, crosslinkers biologically active species and coating thicknesses.

Stents coated and cured in the manner described need to be sterilized prior to packaging for future implantation. For sterilization, gamma radiation is a preferred method particularly for heparin containing coatings; however, it has been found that stents coated and cured according to the process of the invention subjected to gamma sterilization may be too slow to recover their original posture when delivered to a vascular or other lumen site using a catheter unless a pretreatment step as at 24 is first applied to the coated, cured stent.

The pretreatment step involves an argon plasma treatment of the coated, cured stents in the unconstrained configuration. In accordance with this procedure, the stents are placed in—a chamber of a plasma surface treatment system such as a Plasma Science 350 (Himont/Plasma Science, Foster City, Calif.). The system is equipped with a reactor chamber and RI solid-state generator operating at 13.56 MHz and from 0-500 watts power output and being equipped with a microprocessor controlled system and a complete vacuum pump package. The reaction chamber contains an unimpeded work volume of 16.75 inches (42.55 cit) by 13.5 inches (34.3 cm) by 17.5 inches (44.45 cm) in depth.

In the plasma process, unconstrained coated stents are placed in a reactor chamber and the system is purged with nitrogen and a vacuum applied to 20 mTorr. Thereafter, inert gas (argon, helium or mixture of them) is admitted to the reaction chamber for the plasma treatment. A highly preferred method of operation consists of using argon gas, operating at a power range from 200 to 400 watts, a flow rate of 150-650 standard ml per minute, which is equivalent to 100-450 mTorr, and an exposure time from 30 seconds to about 5 minutes. The stents can be removed immediately after the plasma treatment or remain in the argon atmosphere for an additional period of time, typically five minutes.

After this, as shown at 26, the stents are exposed to gamma sterilization at 2.5-3.5 Mrad. The radiation may be carried out with the stent in either the radially non-constrained status or in the radially constrained status.

With respect to the anticoagulant material, heparin, the percentage in the tie layer is nominally from about 30-50% and that of the top layer from about 0-30% active material. The coating thickness ratio of the top layer to the tie layer varies from about 1:6 to 1:2 and is preferably in the range of from about 1:5 to 1:3.

Suppressing the burst effect also enables a reduction in the drug loading or in other words, allows a reduction in the coating thickness, since the physician will give a bolus injection of antiplatelet/anticoagulation drugs to the patient during the stenting process. As a result, the drug imbedded in the stent can be fully used without waste. Tailoring the first day release, but maximizing second day and third day release at the thinnest possible coating configuration will reduce the acute or subcute thrombosis.

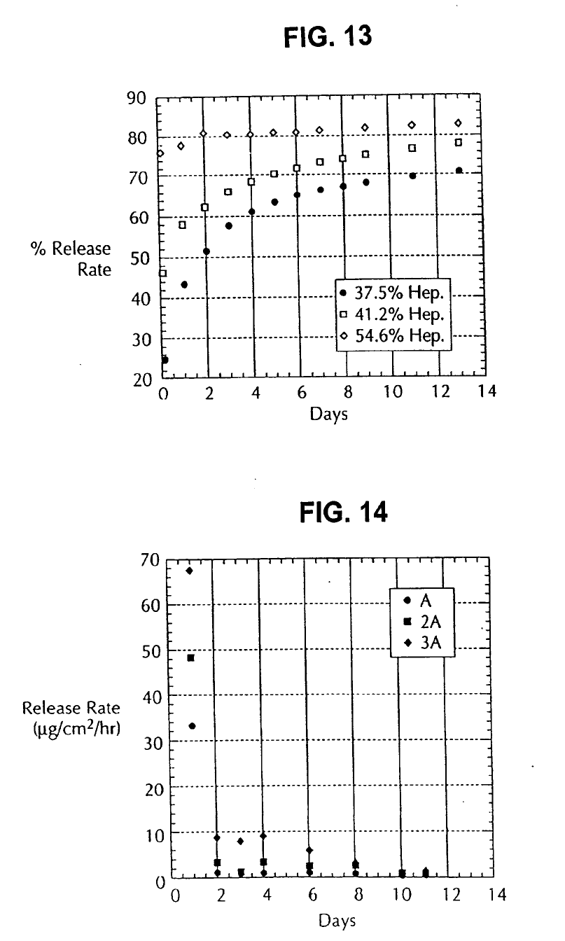

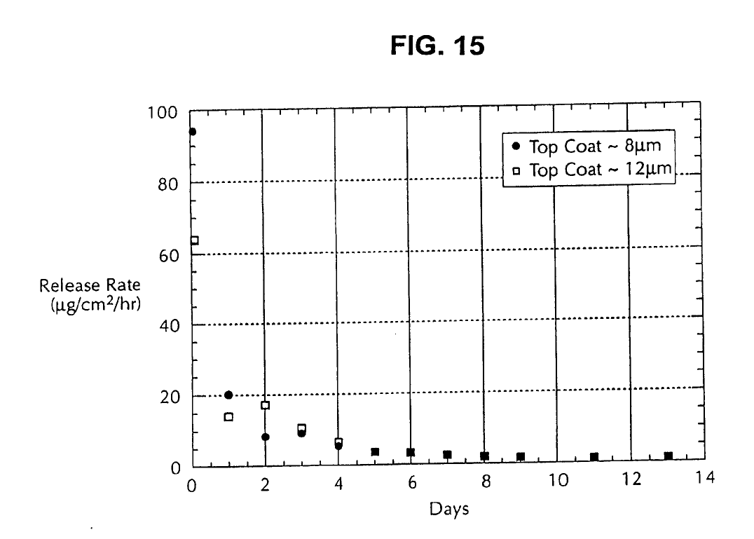

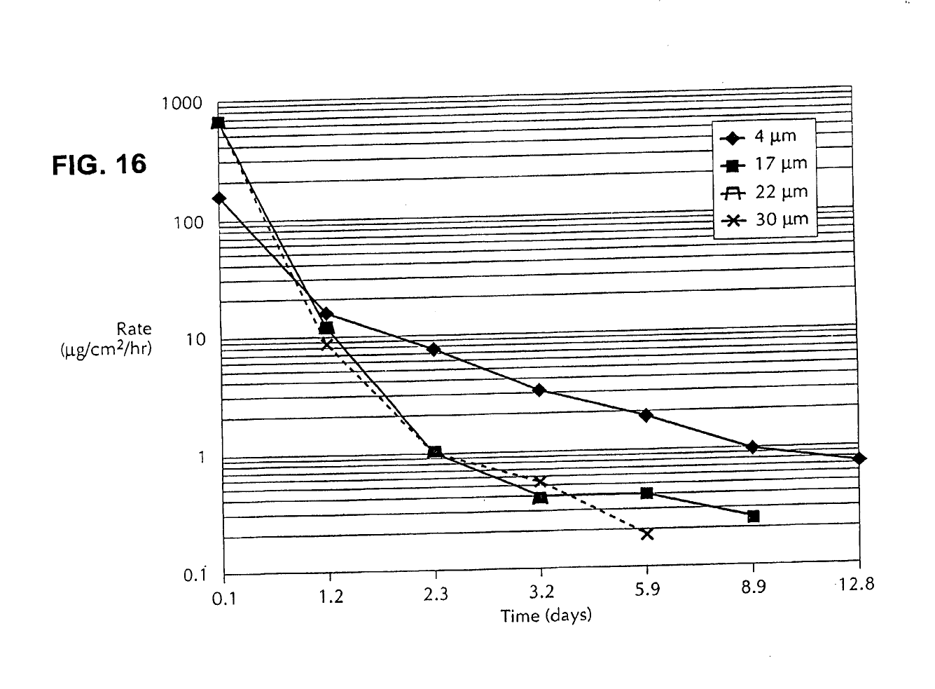

FIG. 13 depicts the general effect of drug loading for coatings of similar thickness. The initial elution rate increases with the drug loading as shown in FIG. 14. The release rate also increases with the thickness of the coating at the same loading but tends to be inversely proportional to the thickness of the top layer as shown by the same drug loading and similar tie-coat thickness in FIG. 15.

What is apparent from the data gathered to date, however, is that the process of the present invention enables the drug elution kinetics to be controlled in a manner desired to meet the needs of the particular stent application. In a similar manner, stent coatings can be prepared using a combination of two or more drugs and the drug release sequence and rate controlled. For example, antiproliferation drugs may be combined in the tie layer and antiplatelet drugs in the top layer. In this manner, the antiplatelet drugs, for example, heparin, will elute first followed by antiproliferation drugs to better enable safe encapsulation of the implanted stent.

The heparin concentration measurement were made utilizing a standard curve prepared by complexing azure A dye with dilute solutions of heparin. Sixteen standards were used to compile the standard curve in a well-known manner.

For the elution test, the stents were immersed in a phosphate buffer solution at pH 7.4 in an incubator at approximately 37° C. Periodic samplings of the solution were processed to determine the amount of heparin eluted. After each sampling, each stent was placed in heparin-free buffer solution.

As stated above, while the allowable loading of the elastomeric material with heparin may vary, in the case of silicone materials heparin may exceed 60% of the total weight of the layer. However, the loading generally most advantageously used is in the range from about 10% to 45% of the total weight of the layer. In the case of dexamethasone, the loading may be as high as 50% or more of the total weight of the layer but is preferably in the range of about 0.4% to 45%.

It will be appreciated that the mechanism of incorporation of the biologically active species into a thin surface coating structure applicable to a metal stent is an important aspect of the present invention. The need for relatively thick-walled polymer elution stents or any membrane overlayers associated with many prior drug elution devices is obviated, as is the need for utilizing biodegradable or reabsorbable vehicles for carrying the biologically active species. The technique clearly enables long-term delivery and minimizes interference with the independent mechanical or therapeutic benefits of the stent itself.

Coating materials are designed with a particular coating technique, coating/drug combination and drug infusion mechanism in mind. Consideration of the particular form and mechanism of release of the biologically active species in the coating allow the technique to produce superior results. In this manner, delivery of the biologically active species from the coating structure can be tailored to accommodate a variety of applications. Whereas the above examples depict coatings having two different drug loadings or percentages of biologically active material to be released, this is by no means limiting with respect to the invention and it is contemplated that any number of layers and combinations of loadings can be employed to achieve a desired release profile. For example, gradual grading and change in the loading of the layers can be utilized in which, for example, higher loadings are used in the inner layers. Also layers can be used which have elutable compounds but no drug loadings at all. For example, a pulsatile heparin release system may be achieved by a coating in which alternate layers containing heparin are sandwiched between unloaded layers of silicone or other materials for a portion of the coating. In other words, the invention allows untold numbers of combinations which result in a great deal of flexibility with respect to controlling the release of biologically active materials with regard to an implanted stent. Each applied layer is typically from approximately 0.5 microns to 15 microns in thickness. The total number of sprayed layers, of course, can vary widely, from less than 10 to more than 50 layers; commonly, 20 to 40 layers are included. The total thickness of the coating can also vary widely, but can generally be from about 10 to 200 microns.

Whereas the polymer of the coating may be any compatible biostable elastomeric material capable of being adhered to the stent material as a thin layer, hydrophobic materials are preferred because it has been found that the release of the biologically active species can generally be more predictably controlled with such materials. Preferred materials include silicone rubber elastomers and biostable polyurethanes specifically.

This invention has been described herein in considerable detail in order to comply with the Patent Statutes and to provide those skilled in the art with the information needed to apply the novel principles and to construct and use embodiments of the example as required. However, it is to be understood that the invention can be carried out by specifically different devices and that various modifications can be accomplished without departing from the scope of the invention itself.

We claim:

1. A method of making a balloon-expandable stent comprising:
providing a metallic intravascular balloon-expandable open lattice sidewall stent structure designed for permanent implantation into a blood vessel of a patient;
applying a first polymer composition to at least a portion of the open lattice sidewall stent structure in a conforming manner so as to preserve the open lattice sidewall stent structure, wherein the first polymer composition comprises a first polymer and a biologically active material; and
applying a second polymer composition conforming to at least a portion of the first polymer composition in a conforming manner so as to preserve the open lattice sidewall stent structure, wherein the second polymer composition comprises a second polymer that is different from the first polymer, and wherein the second polymer composition is substantially free of any biologically active material when applied to the portion of the first polymer composition.

2. The method of claim 1, wherein when in use, the biologically active material is released from the stent to the blood vessel at a first rate that is different from a second rate, wherein the second rate is the rate of release of the same biologically active material from the stent had the second polymer composition not been applied to the first polymer composition.

3. The method of claim 1, wherein the stent comprises stainless steel.

4. The method of claim 1, wherein the first polymer is a biostable polymer.

5. The method of claim 1, wherein the first polymer comprises a hydrophobic biostable elastomeric material.

6. The method of claim 1, wherein the first polymer comprises an ethylene vinyl acetate copolymer material.

7. The method of claim 1, wherein the biologically active material is an agent that inhibits restenosis.

8. The method of claim 7, wherein the agent that inhibits restenosis is a smooth muscle cell inhibitor.

9. The method of claim 1, wherein the biologically active material is an anti-proliferative agent.

10. The method of claim 1, wherein the stent releases the biologically active material over a period of time.

11. A method of making a balloon-expandable stent comprising:
providing a metallic intravascular balloon-expandable open lattice sidewall stent structure designed for permanent implantation into a blood vessel of a patient;
applying a first polymer composition to at least a portion of the open lattice sidewall stent structure in a conforming manner so as to preserve the open lattice sidewall stent structure, wherein the first polymer composition comprises a first biostable polymer and an agent that inhibits restenosis; and
applying a second polymer composition to at least a portion of the first polymer composition in a conforming manner so as to preserve the open lattice sidewall stent structure, wherein the second polymer composition comprises a second biostable polymer that is different from the second biostable polymer, and wherein the second polymer composition is substantially free of any biologically active material when applied to the portion of the first polymer composition.

12. The method of claim 11, wherein when in use, the agent that inhibits restenosis is released from the stent to the blood vessel at a first rate that is different from a second rate, wherein the second rate is the rate of release of the same agent that inhibits restenosis from the stent had the second polymer composition not been applied to the first polymer composition.

13. The method of claim 11, wherein the stent comprises stainless steel.

14. The method of claim 11, wherein the first biostable polymer comprises a hydrophobic elastomeric material.

15. The method of claim 11, wherein the first biostable polymer comprises an ethylene vinyl acetate copolymer material.

16. The method of claim 11, wherein the agent that inhibits restenosis is a smooth muscle cell inhibitor.

17. The method of claim 11, wherein the stent releases the agent that inhibits restenosis over a period of time.

18. A method of making a balloon-expandable stent comprising:
providing a metallic intravascular balloon-expandable open lattice sidewall stent structure designed for permanent implantation into a blood vessel of a patient;
applying a first polymer composition to at least a portion of the open lattice sidewall stent structure in a conforming manner so as to preserve the open lattice sidewall stent structure, wherein the first polymer composition comprises an ethylene vinyl acetate copolymer material and an agent that inhibits restenosis; and
applying a second polymer composition to at least a portion of the first polymer composition in a conforming manner so as to preserve the open lattice sidewall stent structure, wherein the second polymer composition comprises a biostable polymer that is different from the ethylene vinyl acetate copolymer material of the first polymer composition, and wherein the second polymer composition is substantially free of any biologically active material when applied to the portion of the first polymer composition.

19. The method of claim 18, wherein when in use, the agent that inhibits restenosis is released from the stent to the blood vessel at a first rate that is different from a second rate, wherein the second rate is the rate of release of the same agent that inhibits restenosis from the stent had the second polymer composition not been applied to the first polymer composition.

20. The method of claim 18, wherein the stent comprises stainless steel.

21. The method of claim 18, wherein the agent that inhibits restenosis is a smooth muscle cell inhibitor.

22. The method of claim 18, wherein the stent releases the agent that inhibits restenosis over a period of time.
